(19) United States
(12) Patent Application Publication
(10) Pub. No.: US 2023/0398158 A1
(43) Pub. Date: Dec. 14, 2023

(54) NEW COMPOSITION FOR TREATMENT OF SKIN CONDITIONS

(71) Applicant: Lactobio A/S, Copenhagen (DK)

(72) Inventors: Charlotte VEDEL, Copenhagen (DK); Katja Billekop OLSEN, Copenhagen (DK); Søren KJÆRULFF, Copenhagen (DK); Sigurd CHRISTENSEN, Copenhagen (DK)

(73) Assignee: Lactobio A/S, Copenhagen (DK)

(21) Appl. No.: 18/251,657

(22) PCT Filed: Nov. 4, 2021
(86) PCT No.: PCT/EP2021/080640

§ 371 Date: May 3, 2023

(30) Foreign Application Priority Data
Nov. 5, 2020 (DK) PA 2020 01248
Mar. 19, 2021 (DK) PA 2021 00281

(51) Int. Cl. A61K 35/744; A61K 31/194; A61K 31/191; A61K 31/60; A61K 9/00; A61P 17/10

(52) U.S. Cl. CPC A61K 35/744; A61K 31/194; A61K 31/191; A61K 31/60; A61K 9/0014; A61P 17/10

(57) ABSTRACT

The present invention relates to a composition for improvement of a skin condition comprising a supernatant produced from fermentation of a bacteria wherein the supernatant comprises at least 3 metabolites produced by the bacteria during fermentation wherein the at least 3 metabolites include at least one of succinic acid, 2-Hydroxyisocaproic acid, azelaic acid and/or salicylic acid, 5 and wherein at least one of succinic acid, 2-Hydroxyisocaproic acid, azelaic acid, and/or salicylic acid is/are produced by fermentation of a lactic acid bacteria.

### TECHNICAL FIELD OF THE INVENTION

The present invention is related to compositions and use thereof for treatment of skin conditions and, in particular, to topical compositions and use. The present invention is related to compositions that are produced by fermentation of a microorganism. In particular the present invention relates to microbial originating composition useful for modulating a dysfunctional microbiome, preventing a skin condition or treating a skin condition. The present invention relates to a new composition comprising microbial metabolites.

In particular the present invention relates to novel compositions obtained by microorganisms producing at least three functional metabolites by one fermentation.

### BACKGROUND OF THE INVENTION

This invention relates to a composition, a use of said composition and a method for use to prevent or treat skin conditions associated with inflammation, infection, a dysfunctional microbiome, aging, photodamage or scaring. The composition comprising functional metabolites obtained by fermentation of a microorganism, especially supernatant from fermentation of bacteria producing at least three functional metabolites.

Particularly a composition comprising un-purified supernatant from fermentation of bacteria producing functional metabolites.

A number of skin conditions can either cause or be a result of a dysfunctional microbiome, infection, inflammation, damage or any combinations thereof.

Acne is a sebaceous gland abnormality with inflammatory papules, pustules, and cysts and noninflammatory comedos. It mainly afflicts teenagers and young adults, and is associated with pathogenic Cutibacterium acnes (Fitz-Gibbon et al. 2013, J. Invest. Dermatol. 133:2152-60. doi:10.1038/jid.2013.21). Rosacea is a chronic disease of the flush area of the face characterized by a heightened vascular response. It begins as a prominent intermittent flush which becomes permanent followed by telangectasias. Later papules and pustules but no comedos develop. It occurs most commonly in women over 30 years of age. Perioral dermatitis occurs primarily in young women and is characterized by erythema, papules, papulo-vesicles and intermittent eczematous plaques of the chin, nasolabial folds, and upper lip. Itching and burning are often present. The usual treatment consists of antibiotics and corticosteroids. Seborrheic dermatitis is a histopathologically eczematous dermatosis characterized by poorly demarcated scaley erythematous patches with yellowish greasy scales. “Dandruff” is a mild form of this condition localized to the scalp. This disease may involve anyone, several, or all of the following sites: scalp, eyebrows, glabella, paranasal and chin folds, ears and retroauricular sulci, presternal interscapular regions, pubic regions and intergluteal folds. Corticosteroids with tar, sulfur, or antibiotics give temporary control in some cases.

Psoriasis is a common chronic proliferative epidermal disease characterized by keratinocyte epidermal transit time being increased by ninefold. The lesions are sharply demarcated thick erythematous plaques with abundant white scale. The most commonly involved sites include elbows, knees, scalp, genitalia, and gluteal fold. Therapy ranges from topical tar, anthralin, and corticosteroids to systemic methotrexate, psoralens and ultraviolet A light, and ultraviolet B light. Eczematous dermatitis is a pathologic state of epidermal spongiosus that is the end result of a variety of diseases. These include atopic dermatitis, atopic diathesis, allergic and irritant contact reactions, photo allergic and photo toxic reactions, drug eruptions, and severe asteatosis. The site of the eruption depends on the insulting disease. Current therapy consists of topical and systemic corticosteroids and topical tar. The infecting organisms associated with these skin conditions may spread to other skin areas and may even be transmitted to other people and can result in changes of the natural skin microbiome causing a dysfunctional microbiome which further worsen the disease. Present therapy consists of topical and systemic antibiotics.

Common for these conditions is the lack of effective mild treatment, most available treatments are based on hormones or antibiotics having negative side effects and often resulting in an even more dysfunctional microbiome.

Despite many commercially available therapies, especially acne, rosacea and eczema remains a therapeutically challenging condition, with many patients being unresponsive to several attempted therapies, making treatment unpredictable and elusive in many cases.

These are examples of conditions for which the present invention is an effective treatment when applied topically.

Microorganisms were surprisingly identified as during fermentation they will produce metabolites, bacteriocins and organic acids in the desired microbial-inhibiting amounts at a concentration in the supernatant from fermentation which is useful to prevent or treat skin conditions without purification of the single metabolites. Accordingly, some lactic acid bacteria were identified to produce multiple functional metabolites during fermentation including bacteriocins, lactic acid, acetic acid, succinic acid, azelaic acid, salicylic acid, indole-3-lactic acid, indole-3-acetic acid, 2-hydroxybuturic acid, 2-Hydroxyisocaproic acid, N-acetyl tryptophan, glycolic acid, N-acetyl glutamin and/or N-acetylaspartic acid in a supernatant suitable for treatment of skin conditions. The supernatant of the present invention was observed to be suitable for directly use as a skin care product or formulated into a skin care product, in therapeutic or skincare compositions for prevention or treatment of skin conditions or for modulation of dysfunctional microbiomes. The microorganisms of the invention is able to ferment a yield of functional metabolites in fermentation sufficient to provide a broad spectrum antimicrobial activity, anti-inflammatory activity, peeling effects, moisturizing effects or functional effects on skin firmness by activation of fibrillin and collagen synthesis. It was a surprise to identify microorganisms being able to produce multiple functional metabolites in one fermentation, wherein the supernatant is directly useful for topical application without further purification of the single metabolites.

Further it was surprising to determine a synergistic effect between the metabolites and thereby allowing the functional concentration of each metabolite to be lower than the functional concentration needed with a purified metabolite.

Metabolites especially relevant for the present invention is bacteriocins, lactic acid, acetic acid, succinic acid, azelaic acid, salicylic acid, indole-3-lactic acid, indole-3-acetic acid, 2-hydroxybuturic acid, 2-Hydroxyisocaproic acid, N-acetyl tryptophan, glycolic acid, N-acetyl glutamin and N-acetylaspartic acid.

Lactic acid, acetic acid and glycol acid with peeling, exfoliating effects on skin, succinic acid with anti-inflammatory activity, salicylic acid as a peeling agent useful as an anti-acne agent, azelaic acid for treatment of acne and rosacea, however, unknown that these can be produced by a fermentation process in a yield with a functional effect. Indole-3-lactid acid and indole-3-acetic acid being known as an inflammatory component, 2-hydroxybuturic acid known as a moisturizing agent for dry skin and eczema, N-acetyl tryptophan to reduce wrinkles, N-acetyl glutamin as antioxidant and with effect on the biosynthesis of collagen, N-acetylaspartic acid (NAA) known for activation of fibrillin and collagen synthesis and useful as an anti-aging component and for improvement of skin firmness (Gillbro J M, Merinville E, Cattley K, Al-Bader T, Hagforsen E, Nilsson M, Mavon A. Int J Cosmet Sci. 2015 October; 37 Suppl 1:41-6. doi: 10.1111/ics.12250.PMID: 26132508).

Bacteriocins and some organic acids e.g. lactic acid, acetic acid, succinic acid and 2-Hydroxyisocaproic acid are known for antimicrobial activity.

Antimicrobial activity of organic acids is obtained at high concentration of acid which limits the use on skin of mammals as these high acid concentrations can cause significant damage to the skin.

The invention involves a composition comprising the supernatant with active ingredients produced by fermentation and without viable cell material. The use of such composition to prevent or treat skin conditions. Synergy is obtained by the compositions as the effect on skin conditions is obtained at much lower concentration of the single metabolites than the concentration needed if the metabolite is used as a single ingredient. E.g. azelaic acid is used for rosacea in concentration high between 2-20% (w/w), using azelaic acid in a composition comprising multiple metabolites all contributing to a synergistic functional effect and thereby the concentration in use can be significantly reduced, also reducing side effects and any toxic or irritation which can be observed while using these metabolites in the high concentrations >2% (w/w).

### SUMMARY OF THE INVENTION

Thus, an object of the present invention relates to a supernatant from isolated Lactic acid bacteria comprising metabolites effective against skin conditions.

The invention relates to a supernatant comprising active ingredients produced by fermentation.

The active ingredients are functional acids and bacteriocins.

In particular the invention relates to a supernatant comprising least 2 organic acids and at least 1 fermentation by-product.

In particular, it is an object of the present invention to provide a composition that solves the above mentioned problems of the prior art.

In one aspect of the invention the composition is for treatment or prevention of acnes vulgaris, rosacea, atopic dermatitis, eczema or psoriasis.

In one aspect of the invention the composition is for treatment or prevention of skin aging, photo damaging, striae or scaring.

One aspect of the invention the organic acid is selected from; azelaic acid, 2-Hydroxyisocaproic acid, salicylic acid, indole-3-lactic acid, 2-hydroxybuturic acid, N-acetylaspartic acid, succinic acid and lactic acid.

One aspect of the invention the active ingredient is a bacteriocin.

Thus, one aspect of the invention relates to a composition comprising a bacteriocin and at least one organic acid.

Thus, one aspect of the invention relates to a composition comprising a plantaricin and at least one organic acid.

In yet another aspect of the invention at least 2 different bacteriocins are present in the composition.

The composition of the inventions comprises at least the following components: bacteriocin and organic acid wherein the organic acid is salicylic acid and/or lactic acid and/or 2-Hydroxyisocaproic acid and/or azelaic acid and/or succinic acid and/or indole-3-lactic acid and/or 2-hydroxybuturic acid and/or N-acetylaspartic acid and/or indole-3-acetic acid.

In another aspect of the invention the supernatant comprises 2 different bacteriocins produced from fermentation of one lactic acid bacteria, wherein the lactic acid bacteria are not genetically modified to produce bacteriocins.

And in still another aspect of the invention the composition is for treatment or prevention of skin conditions.

The present invention will now be described in more detail in the following.

### DETAILED DESCRIPTION OF THE INVENTION

### Definitions

Prior to discussing the present invention in further details, the following terms and conventions will first be defined:

The term “Skin condition” refers to conditions caused by infection or inflammation, including conditions involving inflammation of the adnexa, dermis and epidermis including such dermatoses as rosacea, acne vulgaris, perioral dermatitis, eczema, seborrheic dermatitis, psoriasis, and tinea cruris as well as microbial infection caused by pathogenic bacteria, yeast, fungi or mites.

The term “bacteriocin” refers to an antimicrobial peptide or protein produced by a bacteria that is active against microorganisms but does not harm the producing bacteria. For purposes of the present invention, bacterocins or bacterocin sources generally include antimicrobial agents suitable for use in formulations as cosmetics or pharmaceuticals. Especially preferred antimicrobial agents include “lantibiotics” (i.e., polypeptides containing lanthionine and beta-methyl lanthionine). Non-limiting examples of such lantibiotics are nisin, such as nisin A or nisin Z, or nisin analogs or related lanthionine-containing peptides, such as pediocin, lactosin, lactacins (e.g., lacticin A, lacticin B, lactacin F), camocin, enterocin, plantaricin, subtilin, epidermin, cinnamycin, duramycin, ancovenin, Pep 5, and the like, individually or in any combination thereof. Other bacterocins that are useful in the present invention include, for example, lactococcins (e.g., lactococcin A, lactococcin B, lactococcin M), leucocoin, helvetican, acidophilucin, caseicin, and the like, individually or in any combination.

The term “plantaricin” refers to bacteriocins from Lactobacillus plantarum, the major types of plantaricins includes Plantaricin A, Plantaricin E, Plantaricin F, Plantaricin J, Plantaricin K, Plantaricin C, Plantaricin D, Plantaricin W, Plantaricin T and Plantaricin S. As well as other plantaricins e.g. Plantaricin35d, Plantaricin MG, Plantaricin 423, Plantaricin 154, Plantaricin 149, Plantaricin 163, Plantaricin LC74, Plantaricin K25, Plantaricin ST31, Plantaricin SA6. In particular broad spectrum Plantaricins e.g. Plantaricin F, Plantaricin DL3, Plantaricin ZJ008, Plantaricin MG, Plantaricin Q7, Plantaricin KL-1Y, Plantaricin 163, Plantaricin 154.

As used herein, the term “fermentation” means lactic acid fermentation, that is, the enzymatic decomposition of carbohydrates to form considerable amounts of lactic acid and/or other organic acids. The term “lactic acid bacteria” includes species from the families Lactobacillaceae, Aerococcaceae, Bifidobacteriaceae, Carnobacteriaceae, Enterococcaceae, Leuconostocaceae and Streptococcaceae. These are considered non-pathogenic and are used as probiotic bacteria in general to improve gastrointestinal flora and in the treatment of gastrointestinal symptoms.

The term “supernatant” refers to the fermentation broth from fermentation of a lactic acid bacteria. The supernatant can be crude comprising both fermentation products, substrate from fermentation broth as well as cell material.

The term “Cell Free Supernatant” refers to the supernatant where live cells has been removed. Cell Free Supernatant (CFS) comprises less than 1000 viable CFU/ml. CFS can comprise cell material from dead cells.

A “fermentation byproduct” may include at least one member chosen from a group comprising sorbate, propionate, benzoate, lactate, acetate and/or include at least one antimicrobial lactic acid producing bacteria metabolite chosen from a group comprising phenyllactic acid, 3-hydroxy phenyllactic acid, 4-hydroxy phenylactic acid, 3-hydroxy propan aldehyde, 1,2 propandiol, 1,3 propandiol, hydrogen peroxide, ethanol, carbon dioxide, carbonic acid, propanoic acid, butyric acid, cyclic dipeptides, cyclo(L-Phe-L-Pro), cyclo(L P-Traps-4-OH-L-Pro), 3-(R)-hydroxydecanoic acid, 3-hydroxy-5-cic dodecanoic acid, 3-(R)-hydroxy dodecanoic acid, and 3-(R)-hyroxytetradecanoic acid. A fermentation byproduct can also be cell wall material from the fermentation microorganism including lipotechoic acid, teichonic acid, peptidoglycan, musin, extracellular polysaccharides and proteins associated to the cell wall.

According to yet another embodiment of the present teachings, the fermentation byproduct includes at least one bacteriocin that is a lantibiotic and/or a non-lantibiotic. According to another embodiment of the present teachings, the fermentation byproduct includes at least one further bacteriocin selected from a group comprising nisin A, nisin Z, nisin Q, nisin F, nisin U, nisin U2, salivarcin X, lacticin J46, lacticin 481, lacticin 3147, salivarcin A, salivarcin A2, salivarcin A3, salivarcin A4, BHT-Aa, BHT Ab, salivarcin A5, salivarcin B, streptin, salivaricin A1, streptin, streptococcin A-FF22, mutacin BNY266, mutacin 1140, mutacin K8, mutacin II, smbAB, bovicin HJ50, bovicin HC5, macedocin, leucocin C, sakacin 5X, enterocin CRL35/mundticin, avicin A, mundticin I, enterocin HF, bavaricin A, ubericin A, leucocin A, mesentericin Y105, sakacin G, curvacin A/sakacin A, lactocin 5, cyctolysin, enterocin A, divercin V41, divercin M35, bavaricin, coagulin, pediocin PA-1, mundticin, piscicocin CS526, piscicocin 126/Vla, sakacin, Pcarnobacteriocin BM1, enterocin P, piscicoin Vlb, penocin A, bacteriocin 31, bacteriocin RC714, hiracin JM79, bacteriocin T8, enterocin SE-K4, carnobacteriocin B2 and Plantaricins.

The present invention relates to supernatant from bacterial strains that are viable and/or are dead (killed), both forms may be included within the scope of the present invention.

In a preferred embodiment of the invention the bacteria is inactivated (killed) after fermentation.

Suitable methods for killing (e.g., biological, chemical or physical killing methods) are sufficiently familiar to those skilled in the art. The killed forms of the microorganisms can include the supernatant and any present metabolites in a lysate ferment composition.

The terms “killed” or “dead” relates to inactivated lactic acid bacteria incapable of cell division and without any metabolic activity. Dead or killed lactic acid bacteria may have intact or ruptured cell membranes and cell walls.

“Lysates”, “derivatives”, “analogues”, “fractions” or “extracts” may be obtained from dead or killed lactic acid bacteria. These lysates, fractions, derivative, analogues, and extracts preferably have the properties of being able to bind or co-aggregate with pathogenic bacteria thereby preventing growth and/or biofilm formation of a pathogen, where “lysate” as well as the term “extract” refers in particular to a solution or suspension in an aqueous medium of the cells of the microorganism according to the invention and comprises, for example, macromolecules such as DNA, RNA, proteins, peptides, lipids, carbohydrates, etc. as well as cell detritus. The lysate preferably includes the cell wall or cell wall constituents including binding receptors for co-aggregation. Methods of producing lysates are sufficiently well known to those skilled in the art and includes, for example, the use of a “French press” or enzymatic lysis, a ball mill with glass beads or iron beads. Cells can be broken open by enzymatic, physical or chemical methods. Examples of enzymatic cell lysis may include individual enzymes as well as enzyme cocktails, for example, proteases, proteinase K, lipases, glycosidases; chemical lysis may be induced by ionophores, detergents such as SDS, acids or bases; physical methods may also be implemented by using high pressures such as the French press, osmolarities, temperatures or alternating between heat and cold. Furthermore chemical, physical and enzymatic methods may of course be combined.

In a preferred embodiment the composition and/or the supernatant according to the present invention is suitable for treating; alleviating, suppressing; prophylaxis a disease associated with a pathogenic microorganism infection in a mammal.

The present invention relates to a methods to control a skin problem caused by a microorganism.

The present invention also relates to a method to inhibit Cutibacterium acnes on the skin of a patient with acnes infection.

The term “inhibition” or “inhibit” as used herein, means the killing of a microorganism, such as an undesired bacteria, or the control of the growth of said microorganism.

A “therapeutic effective amount” of a compound with respect to the subject method of treatment refers to an amount of the compounds in a preparation which, when administered as part of a desired dosage regimen (to a mammal, preferably a human) alleviates a symptom, ameliorates a condition, or slows the onset of disease conditions according to clinically acceptable standards for the disorder or condition to be treated or the cosmetic purpose, e.g., at a reasonable benefit risk ratio applicable to any medical treatment.

As used herein, the term “treating” or “treatment” includes reversing, reducing, or arresting the symptoms, clinical signs, and underlying pathology of a condition in a manner to improve or stabilize a subject's condition.

It will be clear to those skilled in the art that here, as well as in all the statements of range given in the present invention, characterized by such terms as “about” or “approximately,” that the precise numerical range need not be indicated with expressions such as “about” or “approx.” or “approximately,” but instead even minor deviations up or down with regard to the number indicated are still within the scope of the present invention.

A “mammal” include, but are not limited to, humans, primates, farm animals, sport animals, rodents and pets. Non-limiting examples of non-human animal subjects include rodents such as mice, rats, hamsters, and guinea pigs; rabbits; dogs; cats; sheep; pigs; piglets; sows; poultry; turkeys; broilers; minks; goats; cattle; horses; and non-human primates such as apes and monkeys.

In another aspect, the present teachings disclose a fermented composition substantially free from viable microorganisms. The composition can comprise cell material including dead cells.

In one aspect of the invention the composition comprises the supernatant from fermentation and cell material having a further functional effect.

In one aspect of the invention the cell material is capable of co-aggregating with a skin pathogen.

In one aspect of the invention the cell material is capable of co-aggregating with Cutibacterium acnes or Staphylococcus aureus.

In some embodiments, the skin condition is acne, contact dermatitis, actinic dermatitis, dermatitis caused by microbial infection, eczema, or rosacea. In preferred embodiments, the skin condition is acne. In some embodiments, the skin is the skin of the face, scalp, neck, chest, or back. In preferred embodiments, the skin is the skin of the face.

The composition for topical application may preferably be formulated into emulsion, a mist, a paste; a talc; a powder; a lotion; a custard; a foam; a cream; an oil, a serum, an ointment, a spray or semi-solid formulation.

The preferred pH of the supernatant will be pH 2.5 to pH 7, more preferred from pH 3 to pH 6, and even more preferred from pH 3 to pH 5.5. The low pH of the supernatant resulting from the acids produced by fermentation will when applied on skin cause acidification of the surface of skin with elevated pH. Healthy skin has a pH at about 4.5 and the metabolites produced by fermentation will as another beneficial effects assist in maintaining a healthy pH of the skin.

This invention is based upon the discovery that some species of lactic acid bacteria will produce bacteriocin in the supernatant in an amount effective to inhibit growth of skin pathogens even though the lactic acid bacteria are no longer present and there will be no fermentation on the skin.

According to the invention, preferred bacteriocin-producing lactic acid bacteria are Lactobacillus plantarum, Lactobacillus acidophilus, Lactobacillus reuteri, Lactobacillus casei, Lactobacillus johnsonii, Lactobacillus rhamnosus, Lactobacillus gasseri, Bifidobacterium lactis, Bifidobacterium infantis, Bifidobacterium longum, Saccharomyces boulardii, Lactobacillus salivarus, Bacteroides spp, Enterococcus faecium, Lactobacillus delbrucekii spp bulgaricus, Lactobacillus cellibiosus, Lactobacillus curvatus, Lactobacillus brevis, Bifidobacterium bifidum, Bifidobacterium adolescsents, Bifidobacterium animalis, Bifidobacterium thermophilium, Enterococcus faecalis, Streptococcus cremoris, Streptococcus salivarius, Streptococcus diacetylactis, Streptococcus intermedius, Lactobacillus paracasei, Streptococcus thermophiles, Streptococcus salivarius subsp. Thermophilus, Bacillus cereus, Proprionibacteria freundenreichii, Bacillus coagulans (L. sporegenes), Oxalobacter formagenes, Bifidobacterium bifidus, or Leuconostoc mesenteroides, more preferably Lactobacillus plantarum.

In one embodiment of the invention the preferred microorganism is an isolated wild type lactic acid bacteria.

In one embodiment of the invention the preferred microorganism is a gene modified bacteria engineered to be able to produce the metabolites of the present invention in one fermentation.

According to the invention, the preferred lactic acid bacteria is L. plantarum being able to produce at least 0.5% (w/w) of at least one of the following metabolites succinic acid, azelaic acid, salicylic acid, 2-Hydroxyisocaproic acid, indole-3-lactic acid, indole-3-acetic acid, 2-hydroxybuturic acid, N-acetyl tryptophan, glycolic acid, N-acetyl glutamin and/or N-acetylaspartic acid in the supernatant during fermentation.

In an embodiment of the present invention the lactic acid bacteria according to the present invention, in particular L. plantarum, is being able to produce at least 0.05% (w/w), such as at least 0.1% (w/w), e.g. at least 0.25% (w/w), such as at least 0.5% (w/w) of at least one of, preferably, at least two of, more preferably at least 3 of, even more preferably at least 4 of, even more preferably at least 5 of the following metabolites succinic acid, azelaic acid, salicylic acid, 2-Hydroxyisocaproic acid, indole-3-lactic acid, indole-3-acetic acid, 2-hydroxybuturic acid, N-acetyl tryptophan, glycolic acid, N-acetyl glutamin and/or N-acetylaspartic acid, in the supernatant during fermentation. Preferably the metabolites produced in mentioned amounts includes succinic acid, azelaic acid, salicylic acid, and/or 2-Hydroxyisocaproic acid.

The composition according to the present invention have surprisingly found capable of the improvement of a skin condition. The composition comprising a supernatant produced from fermentation of a bacteria wherein the supernatant comprises at least 3 metabolites produced by the bacteria during fermentation.

In an embodiment of the present invention the at least 3 metabolites may include at least one of succinic acid, 2-Hydroxyisocaproic acid, azelaic acid and/or salicylic acid, such as at least 2 of the metabolites, e.g. the at least 3 metabolites include succinic acid, 2-Hydroxyisocaproic acid, azelaic acid and salicylic acid. Preferably, the succinic acid, 2-Hydroxyisocaproic acid, azelaic acid and/or salicylic acid is/are produced by fermentation of a lactic acid bacteria.

The supernatant may be produced from a single fermentation or from a mixture of supernatants obtained from several fermentations.

When the supernatant is provided from a single fermentation, the single fermentation may have resulted in a supernatant comprising at least two of the following metabolites; lactic acid, acetic acid, succinic acid, azelaic acid, salicylic acid, indole-3-lactic acid, 2-Hydroxyisocaproic acid, indole-3-acetic acid, 2-hydroxybuturic acid, N-acetyl tryptophan, glycolic acid, N-acetyl glutamin and N-acetylaspartic acid, such as at least 3 of the metabolites, e.g. at least 4 of the metabolites, such as at least 5 of the metabolites, e.g. at least 6 of the metabolites, such as at least 7 of the metabolites, e.g. at least 8 of the metabolites.

The supernatant may be produced from a mixture of two or more supernatants from various fermentations. In this case each of the supernatants mixed may comprise one or more or the following metabolites; lactic acid, acetic acid, succinic acid, azelaic acid, salicylic acid, indole-3-lactic acid, 2-Hydroxyisocaproic acid, indole-3-acetic acid, 2-hydroxybuturic acid, N-acetyl tryptophan, glycolic acid, N-acetyl glutamin and N-acetylaspartic acid, such as at least 3 of the metabolites, e.g. at least 4 of the metabolites, such as at least 5 of the metabolites, e.g. at least 6 of the metabolites. Preferably, the supernatants mixed may not comprise the same metabolites or same concentration of the various metabolites.]

Bacteriocins are generally known as being effective in inhibiting pathogenic and spoilage microorganisms in foods, such as described by Twomey, D. et al., Lantabiotics Produced by Lactic Acid Bacteria: Structure, Function and Applications, Antonie van Leeuwenhoek, 82:15-185, 2002, and Cleveland, J., et al., “Bacteriocins: Safe, Natural Antimicrobials for Food Preservation,” Int'l J. Food Micro., 71 (2001) 1-20. Bacteriocins are generally understood to act on sensitive cells by forming pores in the cytoplasmic membrane. This leads to the dissipation of the proton motive force and release of small intracellular molecules like glutamate and ATP, such as described by Twomey et al. and Cleveland et al., referenced above. This renders the cells permeable but still capable of participating in biochemical processes in its environment. The treatment of cells with surface-active agents to help generate such “leaky” cells, has been described in PCT Int'l Publication No. WO 01/47366 A1. This activity is typically obtained by the purified bacteriocins or by co-growth of the bacteriocin producing bacteria with the pathogenic and spoilage microorganisms.

In the present invention, at least one secondary antimicrobial agent is included in the supernatant in combination with the bacterocin. Examples of such secondary antimicrobial agents include, for example, metal chelating agents (e.g., citric acid, and the like), organic acids, short chain free fatty acids, proton ionophores (e.g., sorbic acid, benzoic acid, and the like), lacto-antimicrobials (e.g., lactoferrin, lactolipids, and the like), monoglycerides (e.g., monolinolenin, monolaurin, and the like), hops acids, and the like. When used, these secondary antimicrobial agents are generally present at levels of about 0.01 to about 0.5 percent. For organic acids the concentration in the CFS is higher at about 0.5 to 7%.

Especially preferred combinations include bacteriocin and organic acids.

A fermentation byproduct may include at least one member chosen from a group comprising bacteriocins, plantaricins, hydrogen peroxide, lipoteichonic acids, teichonic acids, salts, glycoprotein, and acid mucin.

According to one embodiment of the present teachings, the fermentation byproduct includes at least one antimicrobial lactic acid producing bacterial metabolite chosen from a group comprising 2-Hydroxyisocaproic acid, phenyllactic acid, 3-hydroxyphenyllactic acid, 4-hydroxyphenylactic acid, 3-hydroxy propanaldehyde, 1,2 propandiol, 1,3 propandiol, hydrogen peroxide, ethanol, acetic acid, carbon dioxide, carbonic acid, propanoic acid, butyric acid, cyclic dipeptides, cyclo(L-Phe-L-Pro), cyclo(L P-Traps-4-OH-L-Pro), 3-(R)-hydroxydecanoic acid, 3-hydroxy-5-cic dodecanoic acid, 3-(R)-hydroxy dodecanoic acid, and 3-(R)-hyroxytetradecanoic acid.

According to another embodiment of the present teachings, the fermentation byproduct include at least one bacteriocin that is a lantibiotic (Class II) or a non-lantibiotic (Class II). According to yet another embodiment of the present teachings, the fermentation byproduct include at least one bacteriocin selected from a group comprising Plantaricin A, Plantaricin E, Plantaricin F, Plantaricin J, Plantaricin K, Plantaricin C, Plantaricin D, Plantaricin W, Plantaricin T, Plantaricin S, Plantaricin35d, Plantaricin MG, Plantaricin 423, Plantaricin 154, Plantaricin 149, Plantaricin 163, Plantaricin LC74, Plantaricin K25, Plantaricin ST31, Plantaricin SA6. In particular broad spectrum Plantaricins e.g. Plantaricin F, Plantaricin DL3, Plantaricin ZJ008, Plantaricin MG, Plantaricin Q7, Plantaricin KL-1Y, Plantaricin 163, Plantaricin 154, nisin A, nisin Z, nisin Q, nisin F, nisin U, nisin U2, salivarcin X, lacticin J46, lacticin 481, lacticin 3147, salivarcin A, salivarcin A2, salivarcin A3, salivarcin A4, salivarcin A5, salivarcin B, streptin, salivaricin A1, streptin, streptococcin A-FF22, BHT-Aa, BHT Ab, mutacin BNY266, mutacin 1140, mutacin K8, mutacin II, smbAB, bovicin HJ50, bovicin HC5, macedocin, plantaricin W, lactocin 5, cyctolysin, enterocin A, divercin V41, divercin M35, bavaricin, coagulin, pediocin PA-1, mundticin, piscicocin CS526, piscicocin 126/Vla, sakacin P, leucocin C, sakacin 5X, enterocin CRL35/mundticin, avicin A, mundticin I, enterocin HF, bavaricin A, ubericin A, leucocin A, mesentericin Y105, sakacin G, plantaricin 423, plantaricin C 19, curvacin A/sakacin A, carnobacteriocin BM1, enterocin P, piscicoin Vlb, penocin A, bacteriocin 31, bacteriocin RC714, hiracin JM79, bacteriocin T8, enterocin, or carnobacteriocin. In one preferred embodiment the CFS comprises at least 2 plantaricins selected from the group; Plantaricin A, Plantaricin E, Plantaricin F, Plantaricin J, Plantaricin K, Plantaricin C, Plantaricin D, Plantaricin W, Plantaricin T, Plantaricin S, Plantaricin35d, Plantaricin MG, Plantaricin 423, Plantaricin 154, Plantaricin 149, Plantaricin 163, Plantaricin LC74, Plantaricin K25, Plantaricin ST31, Plantaricin SA6.

In particular at least one plantaricin selected from the group of broad spectrum Plantaricins e.g. Plantaricin F, Plantaricin DL3, Plantaricin ZJ008, Plantaricin MG, Plantaricin Q7, Plantaricin KL-1Y, Plantaricin 163, Plantaricin 154.

In one embodiment the preferred bacteriocin used in the present invention is from fermentation of one of the following bacteria; Weissella viridescens LB10G (DSM 32906), Lactobacillus paracasei LB113R (DSM 32907), Lactobacillus plantarum LB244R (DSM 32996), Lactobacillus paracasei LB116R (DSM 32908), Lactobacillus brevis LB152G (DSM 32995), Lactobacillus paracasei LB28R (DSM 32994), Enterococcus faecium LB276R (DSM 32997), Leuconostoc mesenteriodes LB349R (DSM 33093), Lactobacillus plantarum LB316R (DSM 33091), Lactobacillus plantarum LB356R (DSM 33094), Lactobacillus plantarum LB312R (DSM 33098); and/or any combinations hereof.

The preferred bacteriocin used in the present invention is Plantaricin F. Plantaricin F is produced by e.g. Lactobacillus plantarum LB244R (DSM32996) and e.g. Lactobacillus plantarum LB356R (DSM33094).

Depository details
Weissella viridescens LB10G—deposited at DSMZ, Inhoffenstraße 7B, 38124 Braunschweig, Germany, on 2018-08-28, under accession number DSM 32906
Lactobacillus paracasei LB113R—deposited at DSMZ, Inhoffenstraße 7B, 38124 Braunschweig, Germany, on 2018-08-28, under accession number DSM 32907
Lactobacillus plantarum LB244R—deposited at DSMZ, Inhoffenstraße 7B, 38124 Braunschweig, Germany, on 2018-12-13, under accession number DSM 32996
Lactobacillus paracasei LB116R—deposited at DSMZ, Inhoffenstraße 7B, 38124 Braunschweig, Germany, on 2018-08-28, under accession number DSM 32908
Lactobacillus brevis LB152G—deposited at DSMZ, Inhoffenstraße 7B, 38124 Braunschweig, Germany, on 2018-12-13, under accession number DSM 32995
Lactobacillus paracasei LB28R—deposited at DSMZ, Inhoffenstraße 7B, 38124 Braunschweig, Germany, on 2018-12-13, under accession number DSM 32994
Enterococcus faecium LB276R—deposited at DSMZ, Inhoffenstraße 7B, 38124 Braunschweig, Germany, on 2018-12-13, under accession number DSM 32997
Leuconostoc mesenteriodes LB349R—deposited at DSMZ, Inhoffenstraße 7B, 38124 Braunschweig, Germany, on 2019-04-10, under accession number DSM 33093
Lactobacillus plantarum LB316R—deposited at DSMZ, Inhoffenstraße 7B, 38124 Braunschweig, Germany, on 2019-04-10, under accession number DSM 33091
Lactobacillus plantarum LB356R—deposited at DSMZ, Inhoffenstraße 7B, 38124 Braunschweig, Germany, on 2019-04-10, under accession number DSM 33094
Lactobacillus plantarum LB312R—deposited at DSMZ, Inhoffenstraße 7B, 38124 Braunschweig, Germany, on 2019-04-10, under accession number DSM 33098

The easiest method for providing the CFS comprising bacteriocin, such as plantaricin, is to dry the CFS containing the bacteriocin after fermentation to produce a powder or a concentrated slurry.

The solid materials can be removed after fermentation by filtration or centrifugation from the growth medium. Low molecular weight compounds can be removed by membrane filtration, particularly reverse osmosis. The bacteriocin is a proteinaceous material and can also be separated from the growth medium by precipitation or by other well known techniques such as reverse osmosis and it can then be dried in a pure form.

In one embodiment of the invention the CFS is concentrated.

Concentrating may include separating an amount of the fluid portion from the fermented growth culture using at least one technique chosen from a group comprising filtering, sedimenting, centrifuging, vacuuming, decanting, drying, freeze drying, spray drying, and evaporating. The method for producing the cell free supernatant may further include drying the fermented cell free supernatant.

In one preferred embodiment the CFS is concentrated by removing water.

In one preferred embodiment the CFS is concentrated 2 times by removing water.

In one preferred embodiment the CFS is concentrated 3 times by removing water.

In one preferred embodiment the CFS is concentrated more than 2 times by removing water.

The bacteriocin is preferably used in the final formulation in an amount between 1 and 1,000,000 Arbitrary Units (AU) of bacteriocin. Once AU of bacteriocin was defined as 5 microliters of the highest dilution of culture supernatant yielding a definite zone of growth inhibition with a lawn of an indicator strain on an agar plate.

The organic acid is preferable used in the final formulation in a concentration by weight from about 0.1 to 20%. E.g. by weight from, from 1 to 5 percent lactic acid and from 1.5 to 3 percent acetic acid. The organic acid is preferable selected from lactic acid, acetic acid, malic acid, tartaric acid, propionic acid and succinic acid.

Succinic acid, salicylic acid, azelaic acid, indole-3-lactic acid, indole-3-acetic acid, 2-hydroxybuturic acid, N-acetyl tryptophan, glycolic acid, N-acetyl glutamin and N-acetylaspartic acid is preferable used in the concentrations from 0.001 to 20% (w/w) in the final topical composition, such as in the range of 0.005 to 15% (w/w), e.g. in the range of 0.0075 to 10 (w/w), such as in the range of 0.01 to 5 (w/w), e.g. in the range of 0.025 to 2 (w/w), such as in the range of 0.05 to 1 (w/w), e.g. in the range of 0.075 to 0.5 (w/w), such as about 0.1% (w/w).

The concentration of 2-Hydroxyisocaproic acid in the CFS is preferable above 5 μg/ml, more preferably above 10 μg/ml, more preferably above 20 μg/ml, more preferably above 30 μg/ml, more preferably above 50 μg/ml, more preferably above 75 μg/ml, more preferably above 100 μg/ml, more preferably above 125 μg/ml, more preferably above 150 μg/ml, more preferably above 175 μg/ml, more preferably above 200 μg/ml.

The concentration of succinic acid in the CFS is preferable above 5 μg/ml, more preferably above 10 μg/ml, more preferably above 20 μg/ml, more preferably above 30 μg/ml, more preferably above 50 μg/ml, more preferably above 75 μg/ml, more preferably above 100 μg/ml, more preferably above 125 μg/ml, more preferably above 150 μg/ml, more preferably above 175 μg/ml, more preferably above 200 μg/ml, more preferably above 500 μg/ml, more preferably above 1000 μg/ml.

The concentration of salicylic acid in the CFS is preferable above 5 μg/ml, more preferably above pg/ml, more preferably above 20 μg/ml, more preferably above 30 μg/ml, more preferably above 50 μg/ml, more preferably above 75 μg/ml, more preferably above 100 μg/ml, more preferably above 125 μg/ml, more preferably above 150 μg/ml, more preferably above 175 μg/ml, more preferably above 200 μg/ml, more preferably above 500 μg/ml, more preferably above 1000 μg/ml.

### PREFERRED EMBODIMENTS OF THE INVENTION

Composition for improvement of a skin condition comprising a supernatant produced from fermentation of a bacteria wherein the supernatant comprises at least 3 metabolites produced by the bacteria during fermentation.

Composition for improvement of a skin condition comprising at least 3 metabolites wherein the at least 3 metabolites are selected from; bacteriocins, lactic acid, acetic acid, succinic acid, azelaic acid, salicylic acid, indole-3-lactic acid, indole-3-acetic acid, 2-hydroxybuturic acid, N-acetyl tryptophan, glycolic acid, N-acetyl glutamin and N-acetylaspartic acid.

Composition for improvement of a skin condition comprising a supernatant produced from fermentation of a bacteriocin producing bacteria wherein same bacteria produces at least two of the following metabolites; lactic acid, acetic acid, succinic acid, azelaic acid, salicylic acid, indole-3-lactic acid, indole-3-acetic acid, 2-hydroxybuturic acid, N-acetyl tryptophan, glycolic acid, N-acetyl glutamin and N-acetylaspartic acid.

Composition for improvement of a skin condition comprising a supernatant produced from fermentation of a bacteriocin producing bacteria wherein same bacteria produces at least two of the following metabolites; succinic acid, azelaic acid, salicylic acid, 2-Hydroxyisocaproic acid, indole-3-lactic acid, indole-3-acetic acid, 2-hydroxybuturic acid, N-acetyl tryptophan, glycolic acid, N-acetyl-glutamine and N-acetylaspartic acid.

Composition for improvement of a skin condition comprising at least two of the following metabolites: succinic acid, azelaic acid, salicylic acid, 2-Hydroxyisocaproic acid, indole-3-lactic acid, indole-3-acetic acid, 2-hydroxybuturic acid, N-acetyl tryptophan, glycolic acid, N-acetyl glutamine or N-acetylaspartic acid wherein the metabolite is produced by fermentation of a lactic acid bacteria.

Composition for improvement of a skin condition comprising indole-3-lactic acid wherein the indole-3-lactic acid is produced by fermentation of a lactic acid bacteria.

Composition for improvement of a skin condition comprising salicylic acid wherein the salicylic acid is produced by fermentation of a lactic acid bacteria.

Composition for improvement of a skin condition comprising 2-Hydroxyisocaproic acid wherein the 2-Hydroxyisocaproic acid is produced by fermentation of a lactic acid bacteria.

Composition for improvement of a skin condition comprising 2-hydroxy-buturic acid wherein the 2-hydroxy-buturic acid is produced by fermentation of a lactic acid bacteria.

Composition for improvement of a skin condition comprising N-acetylaspartic acid wherein the N-acetylaspartic acid is produced by fermentation of a lactic acid bacteria.

Composition for improvement of a skin condition comprising azelaic acid wherein the azelaic acid is produced by fermentation of a lactic acid bacteria.

Composition for improvement of a skin condition comprising succinic acid wherein the succinic acid is produced by fermentation of a lactic acid bacteria.

Composition for improvement of a skin condition comprising indole 3-acetic acid wherein the indole 3-acetic acid is produced by fermentation of a lactic acid bacteria.

Composition for improvement of a skin condition comprising N-acetylglutamine wherein the N-acetylglutamine is produced by fermentation of a lactic acid bacteria.

Composition for improvement of a skin condition comprising azelaic acid and indole-3-lactic acid produced by fermentation of a lactic acid bacteria.

Composition for improvement of a skin condition comprising azelaic acid and salicylic acid produced by fermentation of a lactic acid bacteria.

Composition for improvement of a skin condition comprising 2-Hydroxyisocaproic acid and salicylic acid produced by fermentation of a lactic acid bacteria.

Composition according to any one of the compositions described above wherein the composition further comprises a fermentation by-product.

Composition according to any one of the compositions described above wherein the composition comprises lactic acid and at least one bacteriocin.

Composition comprising bacteriocin, azelaic acid, salicylic acid, indole-3-lactic acid, 2-hydroxybuturic acid and N-acetylaspartic acid

Composition comprising bacteriocin, 2-Hydroxyisocaproic acid, salicylic acid, indole-3-lactic acid, 2-hydroxybuturic acid and N-acetylaspartic acid

Composition according to any of the compositions described above wherein the composition is produced by a single fermentation of an isolated lactic acid bacteria Composition according to any of the compositions described above further comprising at least one antimicrobial bacterial metabolite chosen from a group comprising hydrogen peroxide, phenyllactic acid, 3-hydroxyphenyllactic acid, 4-hydroxyphenylactic acid, 2-Hydroxyisocaproic acid, 3-hydroxy propanaldehyde, 1,2 propandiol, 1,3 propandiol, succinic acid, ethanol, acetic acid, carbonic acid, propanoic acid, butyric acid, cyclic dipeptides, cyclo(L-Phe-L-Pro), cyclo(L P-Traps-4-OH-L-Pro), 3-(R)-hydroxydecanoic acid, 3-hydroxy-5-cic dodecanoic acid, 3-(R)-hydroxy dodecanoic acid, and 3-(R)-hyroxytetradecanoic acid.

Composition according to any of the compositions described above further comprising a portion which includes at least one member chosen from a group comprising water, fermentation byproducts, organic acids, fatty acids, growth medium, culture energy source, buffered solution and/or a bacterial cell mass.

Composition according to any of the compositions described above, wherein the bacteriocin producing bacteria is selected from a group comprising Lactobacillus plantarum, Lactobacillus acidophilus, Lactobacillus reuteri, Lactobacillus casei, Lactobacillus johnsonii, Lactobacillus rahamnosus, Lactobacillus gasseri, Bifidobacterium lactis, Bifidobacterium infantis, Bifidobacterium longum, Saccharomyces boulardii, Lactobacillus salivarus, Bacteroides spp, Enterococcus faecium, Lactobacillus delbrucekii spp bulgaricus, Lactobacillus cellibiosus, Lactobacillus curvatus, Lactobacillus brevis, Bifidobacterium bifidum, Bifidobacterium adolescsents, Bifidobacterium animalis, Bifidobacterium thermophilium, Enterococcus faecalis, Streptococcus cremoris, Streptococcus salivarius, Streptococcus diacetylactis, Streptococcus intermedius, Lactobacillus paracasei, Streptococcus thermophiles, Streptococcus salivarius subsp. Thermophilus, Bacillus cereus, Proprionibacteria freundenreichii, Bacillus coagulans (L. sporegenes), Oxalobacter formagenes, Bifidobacterium bifidus, and Leuconostoc mesenteroides.

Composition according to any of the compositions described above wherein the bacteriocin is a plantaricin.

Topical formulation suited for mammals comprising the composition according to any of the compositions described above.

Topical formulation according to the compositions described above wherein the topical formulation is an emulsion, a mist, a paste; a talc; a powder; a lotion; a custard; a foam; a cream; an oil, a serum or an ointment.

Topical formulation according to the compositions described above wherein the topical formulation is a cosmetical product or a pharmaceutical product.

Use of a composition according to any of the compositions described above for the treatment or prevention of a skin condition.

Use of a composition according to any of the compositions described above for the treatment or prevention of a skin condition wherein the skin condition is an inflammatory or infectious disease or skin damage or a dysfunctional skin microbiome.

Use of a composition according to any of the compositions described above for the treatment or prevention of a skin condition wherein the skin condition is selected from: acne, rosacea, eczema, atopic dermatitis and psoriasis.

Use of a composition according to any of the compositions described above for the treatment or prevention of a skin condition wherein the skin condition is selected from: photodamage, aging, striae and scars.

Process to produce a composition according to anyone of the compositions described above.

All patent and non-patent references cited in the present application, are hereby incorporated by reference in their entirety.

The invention will now be described in further details in the following non-limiting examples.

### EXAMPLES

### Example 1

Growth inhibition was measured by contrast phase microscopy and image analysis using the oCelloscope (BioSense Solution, Denmark). The inhibitory effect of Cell Free Supernatant (CFS) on selected pathogens was measured for and various lactic acid bacteria (LAB) strains was tested according to Fredborg et al. with modifications (Fredborg, M., Andersen, K. R., Jorgensen, E., Droce, A., Olesen, T., Jensen, B. B., et al. (2013) ‘Real-Time Optical Antimicrobial Susceptibility Testing’, Journal of Clinical Microbiology, 51(7), pp. 2047-2053. doi: 10.1128/JCM.00440-13). Overnight culture of the pathogenic test organism was diluted to a concentration of approx. 10⁴ CFU/ml. The overnight culture of LAB (109 CFU/ml) was filtered through a 0.2 μm filter to remove all cells. The CFS was diluted into 75%, 50%, 25% and 10%. A 100 μL aliquot of diluted pathogen cell suspension was mixed with 100 μL undiluted or diluted CFS in 96 well plates. The plate was sealed with oxygen penetrating film cover (Sigma-Aldrich) and incubated in the oCelloScope instrument (BioSense Solution, Denmark) at 37° C. for 18 hours. The pathogen growth is measured every 20 min as segmentation and extraction of surface area (SESA).

The following test pathogens/spoilage microorganisms were used:

Staphylococcus aureus MRSA USA300

CFS from the following lactic acid bacteria with antimicrobial activity were tested:

Lactobacillus plantarum LB244R (DSM 32996), Lactobacillus plantarum LB316R (DSM 33091),
Lactobacillus plantarum LB356R (DSM 33094), Lactobacillus plantarum LB312R (DSM 33098),
Lactobacillus paracasei LB116R (DSM 32908), Lactobacillus paracasei LB113R (DSM 32907),
Lactobacillus paracasei LB28R (DSM 32994), Enterococcus faecium LB276R (DSM 32997),
Leuconostoc mesenteriodes LB349R (DSM 33093), Weissella viridescens LB10G (DSM 32906) and Lactobacillus brevis LB152G (DSM 32995)

The Minimum Inhibitory Concentration (MIC) was determined as the most diluted concentration of CFS still being able to growth inhibit the respective pathogen (table 1).

TABLE 1
MIC of the CFS, lowest concentration of CFS able
to growth inhibit the pathogen (% dilution).

| LAB | S. aureus |
|---|---|
| LB244R | 10% |
| LB316R | 75% |
| LB356R | 10% |
| LB312R | 25% |
| LB113R | 25% |
| LB116R | 25% |
| LB276R | 25% |
| LB349R | 25% |
| LB10G | 10% |
| LB152G | 50% |

### Example 2

Metabolomics were done on 3 different media fermentations. Lactic acid bacteria were grown in different growth media (1: malted barley, 2: wheat, 3: barley, 4: barley short extraction 25 min) based on carbohydrate water extraction 1 hour from either barley or whey at 75° C., sterilized by autoclavation and filtered. Fermented at different conditions (1: 30° C. at 48 hours, 2: 30° C. at 48 hours followed by preservation, 3: 30° C. at 48 hours pH adjusted to 5.5). The supernatant analysed by the semi-polar metabolites method. Sample analysis was carried out by MS-Omics (Vedbæk, Denmark) as follows.

The samples were diluted 10 times in 10 mM ammonium formate with 0.1% formic acid. LC-MS method

The analysis was carried out using a UPLC system (Vanquish, Thermo Fisher Scientific) coupled with a high-resolution quadrupole-orbitrap mass spectrometer (Q Exactive™ HF Hybrid Quadrupole-Orbitrap, Thermo Fisher Scientific). An electrospray ionization interface was used as ionization source. Analysis was performed in negative and positive ionization mode. A QC sample was analysed in MS/MS mode for identification of compounds. The UPLC was performed using a slightly modified version of the protocol described by Catalin et al. (UPLC/MS Monitoring of Water-Soluble Vitamin Bs in Cell Culture Media in Minutes, Water Application note 2011, 720004042en).

### Data Processing

Data was processed using Compound Discoverer 3.1 (ThermoFisher Scientific) and TraceFinder 4.1 (ThermoFisher Scientific).

### Compound Extraction

One compound often gives rise to a signal in more than one mass trace (due to e.g. naturally occurring C13 isotopes, adducts, and/or fragments) a compound will therefore almost always be represented by more than one feature with the same retention time but different masses. The compound extraction performed by Compound Discoverer consists of the following four steps:
1) First, features are extracted from the raw data.
2) The feature detection is followed by grouping of features belonging to the same compound.
3) This additional information (e.g. isotope pattern) is then used together with the accurate mass to determine the molecular formula.
4) The total information collected for each compound are then used in the following identification step.

The analysis was carried out using a Thermo Scientific Vanquish LC coupled to Thermo Q Exactive HF MS. An electrospray ionization interface was used as ionization source. Analysis was performed in negative and positive ionization mode. The UPLC was performed using a slightly modified version of the protocol described by Catalin et al. (UPLC/MS Monitoring of Water-Soluble Vitamin Bs in Cell Culture Media in Minutes, Water Application note 2011, 720004042en). Peak areas were extracted using Compound Discoverer 3.1 (Thermo Scientific). Identification of compounds were performed at four levels; Level 1: identification by retention times (compared against in-house authentic standards), accurate mass (with an accepted deviation of 3 ppm), and MS/MS spectra, Level 2a: identification by retention times (compared against in-house authentic standards), accurate mass (with an accepted deviation of 3 ppm). Level 2b: identification by accurate mass (with an accepted deviation of 3 ppm), and MS/MS spectra, Level 3: identification by accurate mass alone (with an accepted deviation of 3 ppm).

A total of 1606 compounds were detected in the samples. Hereof were 271 annotated on level 3, 103 on level 2b, 113 on level 2a, and 60 on level 1.

Lactic acid, acetic acid, succinic acid, azelaic acid, salicylic acid, indole-3-lactic acid, indole-3-acetic acid, 2-hydroxybuturic acid, 2-Hydroxyisocaproic acid and N-acetylaspartic acid were all annotated at level 1 in significant amounts for the two strains LB356R and LB244R.

Organic acid was in the concentration above 3% (w/w) for all fermentations of LB356R and LB244R.

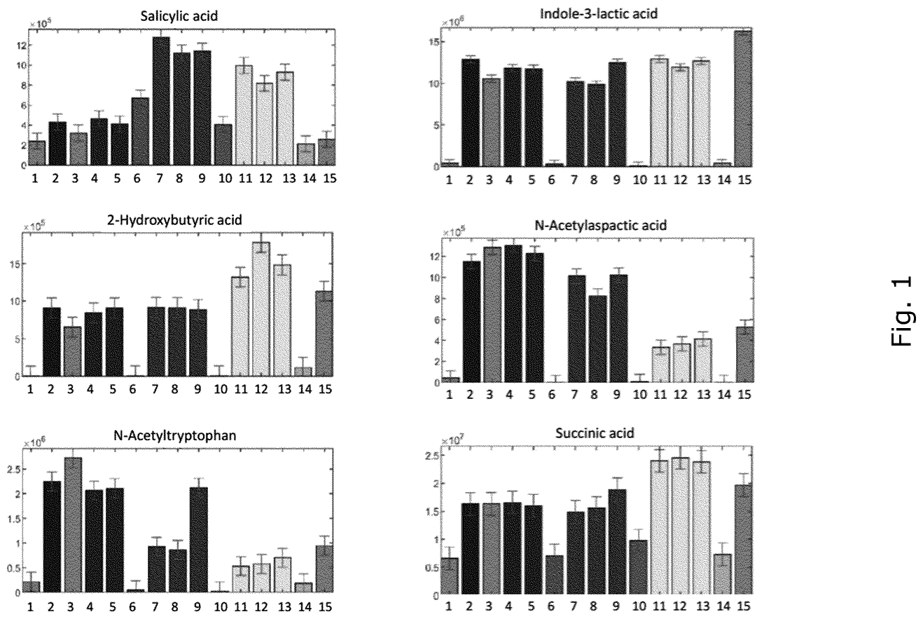

Salicylic acid, indole-3-lactic acid, 2-hydroxybuturic acid and N-acetylaspartic acid is shown relative to control for strain LB356R in FIG. 1. Four different substrates are used for fermentation, for strain LB356R the 4 functional acids are produced in significant amounts in all 4 substrates. The coding of samples shown in FIG. 1 is as follows:

Code for FIG. 1:
1 Control (growth media 1 before fermentation)
2 Growth media 1, fermentation condition1
3 Growth media 1, fermentation condition2
4 Growth media 1, fermentation condition3
5 Growth media 1, fermentation condition3
6 Control (growth media 2 before fermentation)
7 Growth media 2, fermentation condition1
8 Growth media 2, fermentation condition1
9 Growth media 2, fermentation condition1
10 Control (growth media 3 before fermentation)
11 Growth media 3, fermentation condition1
12 Growth media 3, fermentation condition1
13 Growth media 3, fermentation condition1
14 Control (growth media 4 before fermentation)
15 Growth media 4, fermentation condition1

### Example 3

The bacteriocins in the two most active strains were identified by sequencing. Whole genome sequenced by Baseclear (Leiden, Netherlands) and annotated by servers such as Rapid Annotation Subsystem Technology (RAST) server (http://rast.nmpdr.org/) and the annotation program Bacteriocin Genome mining tool, BAGEL4 (http://bagel4.molenrug.nl/index.php) to reveal potential bacteriocin encoding genes and for virulence or disease encoding genes. Subsequently, the genome sequence of LB244R and LB356R was annotated by Baseclear. Several genes involved in bacteriocin production were identified in the LB244R and LB356R genome sequences (Table 2).

| LAB | Identified bacteriocins |
|---|---|
| Lactobacillus plantarum LB244R | Plantaricin E |
| | Plantaricin F |
| | Plantaricin A |
| | Plantaricin J |
| | Enterocin |
| Lactobacillus plantarum LB356R | Plantaricin E |
| | Plantaricin F |
| | Plantaricin A |
| | Plantaricin N |
| | Plantaricin J |
| | Plantaricin K |

### Example 4

Bacterial lysates were produced using 3 different methods.

LAB isolate (LB356R) were grown in MRS broth overnight at 37° C.

Lysate 1: Tube with 15 ml of overnight culture were placed in an ice-batch and cell were lysed by sonication for 30 min using a Q125 sonicator (QSonica).

Lysate 2: 15 ml of overnight culture were adjusted with 0.1M HCl to pH 3 incubated for 2 days at 45 degrees Celsius.

Lysate 3: 15 ml of overnight culture were added 5% SDS (Sigma-Aldrich L3771) and stored at −20 degrees Celsius for 1 hour, incubated at 50 degrees Celsius for 1 hour. Cycles changing temperature from −20 to +50 degrees were done for 2×8 hours Each lysate was evaluated for viable cells after the lysate processes, respectively.

Lysates were evaluated for maintained activity against two skin pathogens:

Staphylococcus aureus MRSA USA300 (ATCC BAA-1717) and Cutibacterium acnes (ATCC ref HM-512) by testing for growth inhibitory activity by co-incubating each lysate 1:1 in BHI inoculated with either HM-512 or USA300 approximately to 10⁴ CFU/ml and follow growth and growth inhibition by measuring OD (600 nm) using a spectrophotometer. All three lysates were shown to growth inhibit both HM-512 and USA300.

### Example 5

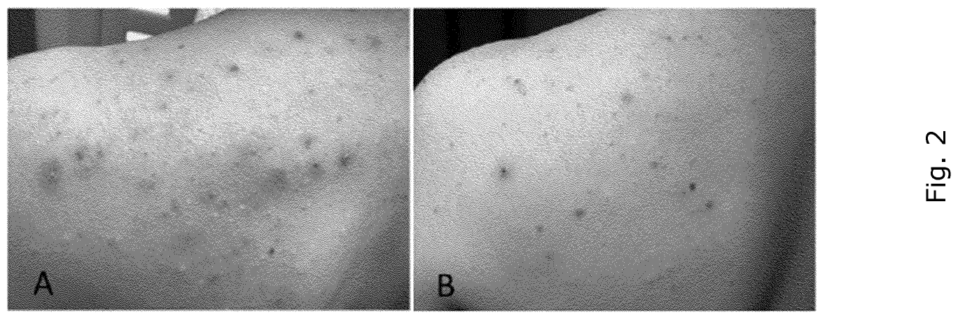

Evaluation test on acne skin was performed by using the supernatant from Lactobacillus plantarum LB356R in a simple water dilution comprising 5% supernatant. Left shoulder of 17 year boy was treated with the supernatant 1 time every day for 14 days. Viable effect on acne was observed after 14 days (FIG. 2). FIG. 2A was before treatment and FIG. 2B was after 14 days.

1.-15. (canceled)

16. Composition for improvement of a skin condition comprising a supernatant produced from fermentation of a bacteria wherein the supernatant comprises at least 3 metabolites produced by the bacteria during fermentation wherein the at least 3 metabolites include at least one of succinic acid, 2-Hydroxyisocaproic acid, azelaic acid and/or salicylic acid, and wherein at least one of succinic acid, 2-Hydroxyisocaproic acid, azelaic acid, and/or salicylic acid is/are produced by fermentation of a lactic acid bacteria.

17. Composition of claim 16 wherein the at least 3 metabolites are selected from; bacteriocins, lactic acid, acetic acid, succinic acid, azelaic acid, salicylic acid, indole-3-lactic acid, indole-3-acetic acid, 2-Hydroxyisocaproic acid, 2-hydroxybuturic acid, N-acetyl tryptophan, glycolic acid, N-acetyl glutamin and N-acetylaspartic acid.

18. Composition for improvement of a skin condition comprising a supernatant produced from fermentation of a bacteriocin producing bacteria wherein same bacteria produces at least two of the following metabolites; lactic acid, acetic acid, succinic acid, azelaic acid, salicylic acid, indole-3-lactic acid, 2-Hydroxyisocaproic acid, indole-3-acetic acid, 2-hydroxybuturic acid, N-acetyl tryptophan, glycolic acid, N-acetyl glutamin and N-acetylaspartic acid.

19. Composition for improvement of a skin condition comprising salicylic acid wherein the salicylic acid is produced by fermentation of a lactic acid bacteria.

20. Composition for improvement of a skin condition comprising succinic acid wherein the succinic acid is produced by fermentation of a lactic acid bacteria.

21. Composition for improvement of a skin condition comprising 2-Hydroxyisocaproic acid wherein the 2-Hydroxyisocaproic acid is produced by fermentation of a lactic acid bacteria.

22. Composition for improvement of a skin condition comprising azelaic acid and salicylic acid, and wherein the azelaic acid and the salicylic acid are produced by fermentation of a lactic acid bacteria.

23. Composition according to any of the proceeding claims further comprising at least one antimicrobial bacterial metabolite chosen from a group comprising hydrogen peroxide, phenyllactic acid, 3-hydroxyphenyllactic acid, 4-hydroxyphenylactic acid, 3-hydroxy propanaldehyde, 1,2 propandiol, 1,3 propandiol, succinic acid, ethanol, acetic acid, carbonic acid, propanoic acid, butyric acid, cyclic dipeptides, cyclo(L-Phe-L-Pro), cyclo(L P-Traps-4-OH-L-Pro), 3-(R)-hydroxydecanoic acid, 3-hydroxy-5-cic dodecanoic acid, 3-(R)-hydroxy dodecanoic acid, 2-Hydroxyisocaproic acid and 3-(R)-hyroxytetradecanoic acid.

24. Composition according to any of the proceeding claims, wherein the bacteriocin producing bacteria is selected from a group comprising Lactobacillus plantarum, Lactobacillus acidophilus, Lactobacillus reuteri, Lactobacillus casei, Lactobacillus johnsonii, Lactobacillus rahamnosus, Lactobacillus gasseri, Bifidobacterium lactis, Bifidobacterium infantis, Bifidobacterium longum, Saccharomyces boulardii, Lactobacillus salivarus, Bacteroides spp, Enterococcus faecium, Lactobacillus delbrucekii spp bulgaricus, Lactobacillus cellibiosus, Lactobacillus curvatus, Lactobacillus brevis, Bifidobacterium bifidum, Bifidobacterium adolescsents, Bifidobacterium animalis, Bifidobacterium thermophilium, Enterococcus faecalis, Streptococcus cremoris, Streptococcus salivarius, Streptococcus diacetylactis, Streptococcus intermedius, Lactobacillus paracasei, Streptococcus thermophiles, Streptococcus salivarius subsp. Thermophilus, Bacillus cereus, Proprionibacteria freundenreichii, Bacillus coagulans (L. sporegenes), Oxalobacter formagenes, Bifidobacterium bifidus, and Leuconostoc mesenteroides.

25. Topical formulation comprising the composition according to any of claims 16-24, wherein the topical formulation is a cosmetical product or a pharmaceutical product.

26. A pharmaceutical product comprising the composition according to anyone of claims 16-24 for the treatment or prevention of a skin condition.

27. The pharmaceutical product according to claim 26, wherein the skin condition is an inflammatory or infectious disease or skin damage or a dysfunctional skin microbiome.

28. The pharmaceutical product according to claim 26, wherein the skin condition is selected from: acne, rosacea, eczema, atopic dermatitis and psoriasis.

29. The pharmaceutical product according to claim 26, wherein the skin condition is selected from: photodamage, aging, striae and scars.

30. Process to produce a composition according to anyone of claims 16 to 24 wherein the process involves fermentation of an isolated lactic acid bacteria.
